(19) United States
(12) Patent Application Publication
(10) Pub. No.: US 2018/0303536 A1
(43) Pub. Date: Oct. 25, 2018

(54) CRYOABLATION SYSTEM

(71) Applicants: Ronny Kafiluddi, Loudonville, NY (US); Ersno Eromo, Ghent, NY (US)

(72) Inventors: Ronny Kafiluddi, Loudonville, NY (US); Ersno Eromo, Ghent, NY (US)

(21) Appl. No.: 15/796,432

(22) Filed: Oct. 27, 2017

Related U.S. Application Data
(60) Provisional application No. 62/413,975, filed on Oct. 27, 2016.

(51) Int. Cl. A61B 18/02

(52) U.S. Cl. CPC A61B 18/02; A61B 2018/00577; A61B 2018/0293

(57) ABSTRACT

A system and method for cryoablation and more particularly, to a cryoprobe needle that can be used accurately to localize and block conduction of a targeted peripheral nerve. In addition, aspects of the disclosure provide a solution for assessing the effectiveness of the cryoablation procedure by repeating an electromyogram (EMG) recording after the treatment. The system can be implemented as a single physical device, capable of generating stimulation pulses, generating extreme temperature changes via a cryoprobe needle, measuring temperature at a tip of an electrode, displaying the measured temperature, maintaining a constant temperature at the tip, and measuring and recording data corresponding to EMG or motor unit action potentials (MUAPs), correlating the data with a location of a target nerve, a treatment being performed on the target nerve, and an effectiveness of the treatment. In addition, aspects of the disclosure provide a solution for assessing the effectiveness of the cryoablation procedure by repeating an electromyogram (EMG) recording after the treatment.

### CROSS REFERENCE TO RELATED APPLICATION

This application claims priority to U.S. Provisional Application Ser. No. 62/413,975, filed on Oct. 27, 2016, entitled CRYOABLATION SYSTEM, the disclosure of which is hereby incorporated by reference in its entirety.

### TECHNICAL FIELD

This disclosure relates generally to a cryoablation system and to a cryoprobe needle that can be used to accurately localize and block conduction of a targeted peripheral nerve.

### BACKGROUND

Nerve pain is often associated with an injury or disease of a peripheral nerve. Nerve pain can lead to chronic pain and can be extremely debilitating. Examples of chronic nerve pain include cranialfacial pain (supra-orbital neuralgia, infra-orbital neuralgia, mandibular neuralgia, trigeminal neuralgia, glossopharyngeal neuralgia etc.), chestwall nerve pain (intercostal neuralgia, postherpetic neuritis etc.), pain in the abdominal or pelvic region (ilioinguinal, iliohypogastric, genitofemoral, punetal neuralgia etc.), sacral pain (sacral neuralgia), or cervical, thoracic and lumbar pain stemming from facet arthrosis.

Patients are diagnosed with chronic nerve pain after a patient history, exam, and possible electrodiagnostic evaluation, which include using nerve conductions studies with an electromyelograph (EMG). Diagnostic blocks (using local anesthetics) are also frequently used. If the pain subsides after blocking a nerve from the painful region, a physician may conclude that the nerve is the source of the pain. However, the precise location of the pain generating nerve is often assumed and hardly confirmed.

Ultrasound guidance has been used in the past to visualize peripheral nerves. However, ultrasound visualization is often limited by patient comorbidities such as obesity or muscular atrophy. Furthermore, ultrasound guidance is inherently limited in the quality of the images if the nerve is deeply positioned or if the nerve is surrounded by similarly dense soft tissue such as ligaments and tendons.

Interventional spine specialists, radiologist, orthopedic surgeons, neurosurgeons, pain management physicians, as well as rehabilitation specialists routinely provide diagnostic and therapeutic injections with the goal of isolating peripheral nerves.

Signal conduction from the nerve, including pain, travels on axons that project from the nerve cells or neurons. Individual axons are surrounded by a myelin sheet and are called myelinated nerves. Myelinated nerves are surrounded by connective tissue (endoneurium). Multiple axons form a fascicle and each fascicle is also supported by connective tissue (perinuerium). A bundle of fascicles is in turn given structural support by an epineurium. Pain signals travel from a painful site and are conducted through bundles of fascicles toward the central nervous system, which comprises the spinal cord and the brain. The interruption of the nerve signal may result in pain relief. Traditionally, the interruption of pain signals are achieved by a chemical modality (e.g. local anesthetics, alcohol, or phenol), or heat therapy (as in radiofrequency ablation).

Current localization and treatment of pain generating nerves can be performed as part of a cryoablation procedure. Cryoablation is a treatment modality that involves freezing the nervous tissue. Cryoablation therapy is premised on the adiabatic cooling of gas (Joule-Thomson effect). As cryogenic substance travels in a closed volume system, a decrease of pressure results in a rapid drop in temperature. The rapid temperature drop subsequently cools the closed volume system and also any tissue in contact with the system. Freezing nerve tissue may reversibly interrupt the conduction of action potentials if the targeted temperatures are controlled between −20 and −100 degrees Celsius. When temperatures are between −20 and −100 degrees Celsius, the nerve axon is temporarily disrupted, resulting in conduction block of an action potential. At those temperatures, however, the perineurium and epineurium remain intact and the nerve retains the ability to regenerate. When frozen at these temperatures, the nerve will initially degenerate at a region distal to the lesion (Wallerian degeneration) but will subsequently regenerate using the endoneurium and perineurim as a scaffolds at a rate of 2-5 mm/day.

When the temperature drop is below −140 degrees Celsius, the nerve tissue may be permanently damaged and the action potential permanently blocked. At temperatures below −140 degrees Celsius, both axons and all surrounding connective tissue, including endoneurium, perineuirum, and epineurium, are permanently damaged, thus preventing the potential for nerve regeneration.

### SUMMARY

Aspects of the present disclosure relate to a cryoablation system and to a cryoprobe needle that can be used accurately to localize and block conduction of a targeted peripheral nerve. In addition, aspects of the disclosure provide a solution for assessing the effectiveness of the cryoablation procedure by repeating an electromyogram (EMG) recording after the treatment.

An embodiment of the present disclosure provides a system, which can be implemented as a single physical device, capable of generating stimulation pulses, generating extreme temperature changes via a cryoprobe needle, measuring temperature at a tip of an electrode, displaying the measured temperature, maintaining a constant temperature at the tip, measuring and recording data corresponding to EMG or motor unit action potentials (MUAPs), and correlating the data with a location of a target nerve, a treatment being performed on the target nerve, and an effectiveness of the treatment.

Other aspects of the present disclosure provide a cryoprobe needle with a unique inner tube for the delivery of anesthetic drugs and an outer tube with chambers that allow for the inflow and exhaust of cryogenic substance. The flow of cryogenic substance allows the tip of the cryoprobe to rapidly cool and thus, through contact with the targeted nerve, can create cryolesions or ice ball that can result in a temporary or permanent nerve block.

Another aspect is A needle for applying cryoablation to a peripheral nerve in a patient, the needle comprising: an insulated needle shaft, the insulated needle shaft comprising: an inner shaft tube; at least two cannula leads; an outer shaft tube, wherein the outer shaft tube comprises an inflow chamber and an exhaust chamber, the inflow chamber configured to allow inflow of a cryogenic substance and the exhaust chamber configured to allow exhaust of the cryogenic substance; and a cryoprobe tip connected to the distal end of the insulated needle shaft, the cryoprobe tip comprising: a conducting surface configured to rapidly cool; at least two apertures connected to the cannula leads; and an opening connected to the inner shaft.

A further aspect is a system for blocking the conduction of a targeted peripheral nerve in a patient, the system comprising: a cryoprobe needle, wherein the cyroprobe needle comprises: an inner channel configured to allow injection of a local anesthetic; an insulated needle shaft with an inflow chamber and an exhaust chamber, the inflow chamber configured to allow inflow of a cryogenic substance and the exhaust chamber configured to allow exhaust of the cryogenic substance; at least two cannula leads; a drug delivery device for the delivery of drug; and a control system for generating electrical stimulation signals and measuring data corresponding to EMG or motor unit action potentials.

Yet another aspect is a method for blocking the conduction of targeted peripheral nerve in a patient, the method comprising: inserting a cryoprobe through a skin of a patient and into a first position toward a targeted peripheral nerve; generating with an electrical stimulator a first signal at a conducting tip of the cryoprobe, wherein the first signal stimulates the targeted peripheral nerve to innervate a targeted muscle; receiving a second signal from the targeted muscle at a recording tip of the cryoprobe, the second signal being associated with muscle activity of the targeted muscle; and generating with the cryoprobe a cryolesion on the targeted peripheral nerve; generating with an electrical stimulator a third signal at a conducting tip of the cryoprobe, wherein the third signal stimulates the targeted peripheral nerve at the location of the cryolesion; and receiving a fourth signal from the muscle at a recording tip of the second needle, the fourth signal confirming successful ablation of the peripheral nerve.

A further aspect is a needle for applying cryoablation to a peripheral nerve in a patient, the needle comprising: an insulated needle shaft, the insulated needle shaft comprising: an inner shaft tube; an outer shaft tube, wherein the outer shaft tube comprises an inflow chamber and an exhaust chamber, the inflow chamber configured to allow inflow of a cryogenic substance and the exhaust chamber configured to allow exhaust of the cryogenic substance; and a cryoprobe tip with a conducting surface and an opening connected to inject drugs.

Other aspects of the present disclosure provide methods using and generating each, which include and/or implement some or all of the actions described herein. The illustrative aspects of the present disclosure are designed to solve one or more of the problems herein described and/or one or more other problems not discussed.

### BRIEF DESCRIPTION OF THE DRAWINGS

These and other features of the disclosure will be more readily understood from the following detailed description of the various aspects of the present disclosure taken in conjunction with the accompanying drawings that depict various aspects of the present disclosure.

FIG. 1 shows an illustrative environment for using a cryoablation system in a patient by a physician according to embodiments of the present disclosure.

FIG. 2 shows an illustrative example of the cyroablation system connected to a schematic diagram of a cryoprobe needle.

FIG. 3 shows a schematic diagram of an example of the cryoprobe needle.

FIG. 4 is a schematic longitudinal cross section of the example cyroprobe needle.

FIG. 5 is a schematic circular cross section of an example cryoprobe needle shaft.

FIG. 6 is a flowchart illustrating an example method of use of the cryoablation system according to embodiments of the present disclosure.

It is noted that the drawings may not be to scale. The drawings are intended to depict only typical aspects, and therefore should not be considered as limiting. In the drawings, like numbering represents like elements between the drawings.

### DETAILED DESCRIPTION

Various embodiments will be described in detail with reference to the drawings, wherein like reference numerals represent like parts and assemblies throughout the several views. Reference to various embodiments does not limit the scope of the claims attached hereto. Additionally, any examples set forth in this specification are not intended to be limiting and merely set forth some of the many possible embodiments for the appended claims.

This disclosure relates generally to a cryoablation system and more particularly to a cryoprobe needle and a cryoablation system that can be used to accurately localize and block conduction of a targeted peripheral nerve. In addition, aspects of the disclosure provide a solution for assessing the effectiveness of the cryoablation procedure by repeating an electromyogram (EMG) recording after the treatment.

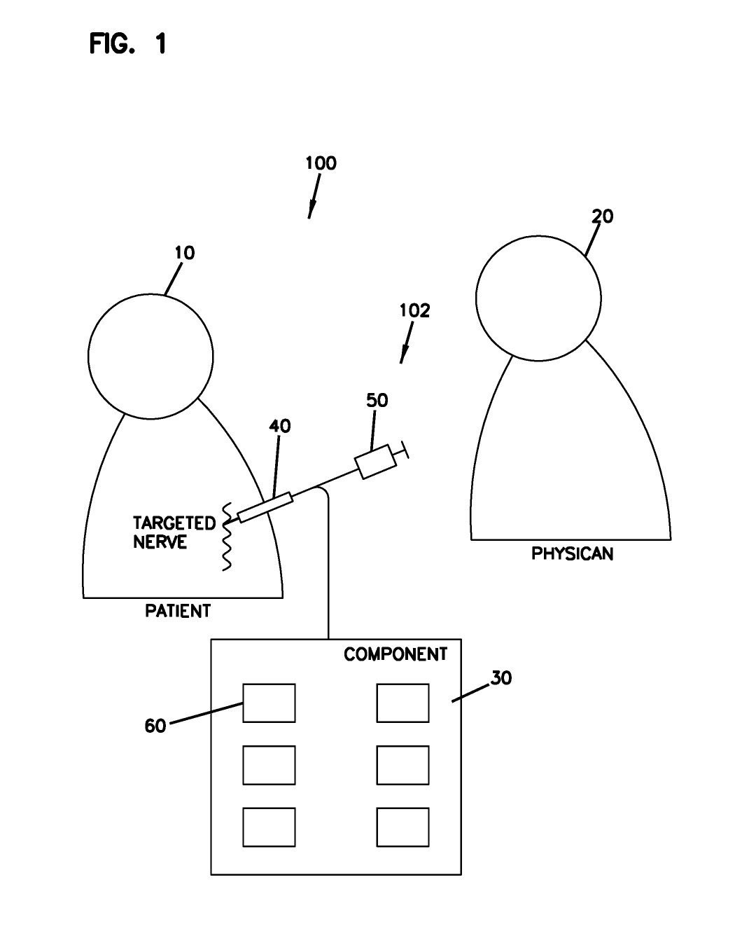

Turning to the drawings, FIG. 1 shows an illustrative environment 100 for locating a targeted nerve in a patient 10 by a physician 20. To this extent, the environment 100 includes a cryoablation system 102 for locating a target peripheral nerve in the patent. In particular, the cryoablation system 102 is shown including a cryoprobe needle 40, a drug delivery device 50, and a control system 30. In some embodiments the control system includes one or more components 60.

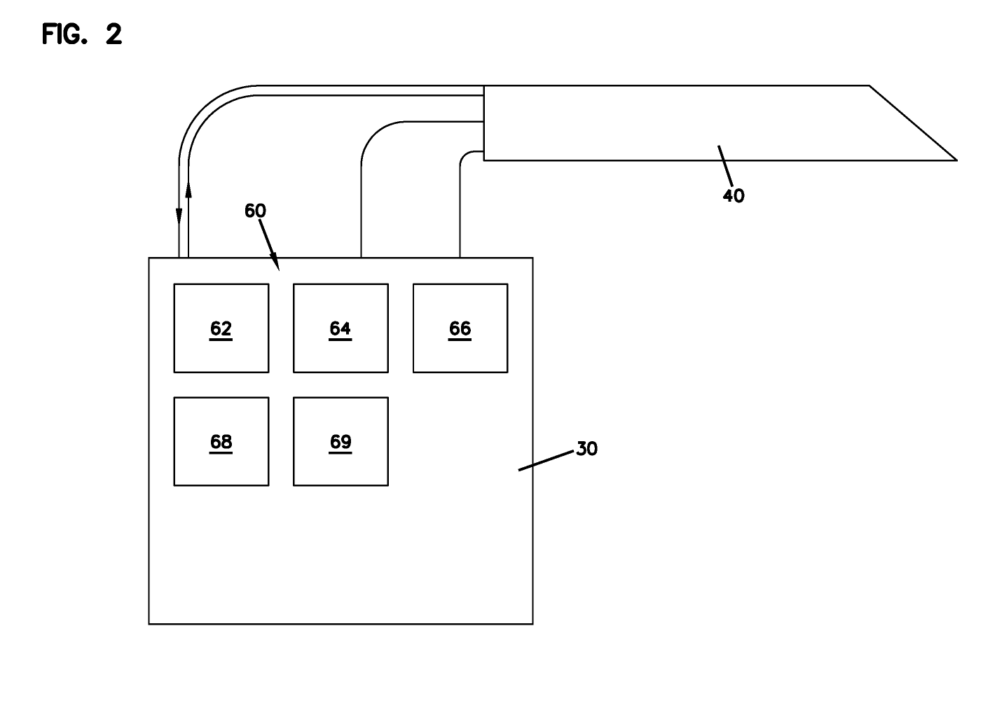

FIG. 2 is a schematic diagram of an example control system 30 connected to an example cryoprobe needle 40. In this example, the control system 30 includes a plurality of components 60. The components 60 can include one or more of: an electrical stimulating component 62, an electrical recording component 64, a display component 66 (e.g., one or more displays), a temperature sensing component 68, and a cryogenic substance delivery component 69. In some embodiment, the control system 30 includes more, fewer, or different components, and may include various combinations of one or more of these or other components.

In general terms, the electrical stimulating component 62 operates to generate electricals signals for delivery to the nerve, thereby causing muscle activity by electrical stimulation. In some embodiments the electrical stimulating component 62 includes a signal generator, which operates to generate electrical signals suitable for nerve and muscle stimulation. In some embodiments the electrical stimulating component 62 is connected to and utilizes one or more wire electrodes to deliver the electrical signals to the patient. Wire electrodes may be made from small diameter, non-oxidizing, stiff insulated wire. The wire may be composed of metal or metal alloys, such as platinum, silver, nickel, or chromium alloy, for example. The insulation may be composed from nylon, polyurethane, or Teflon, or other insulating materials. Teflon and nylon generally provide better mechanical rigidity to the overall wire, thereby assisting in control and placement. A wire electrode is easily implantable and retractable from tissue, and thereby elicits minimal pain from a patient when in use. A needle electrode, in contrast, is much stiffer and is typically inserted in the muscle throughout the duration of the exam and therefore, may elicit a greater pain response.

In general, the electrical recording component 64 operates to detect electromyographic (EMG) signals from a patient. In some embodiments the electrical recording component 64 is an electromyograph. The EMG signals represent neuromuscular activity associated with a contracting muscle. The signal generated from muscle activity represents an electrical current generated by the flow of ions across a membrane of the muscle fibers. The flow eventually reaches to a contact surface of the electrical recording component 64, discussed in further detail herein. The system may involve consideration of a number of factors, including the anatomy and physiology of the muscle as well as the general nature of the nervous system and the specific characteristics of the tools used to detect or record the EMG signal. One of the fundamental units of the EMG is a motor unit action potential (MUAP). The MUAP is an electrical signal that is generated from a muscle cell unit, called a motor unit. As with EMG signals in general, the general strength and characteristic of the MUAP is also affected by the anatomy and physiology of the muscle as well as the general nature of the nervous system and the specific characteristics of the tools used to detect or record the MUAP signal.

Some embodiments include an electrical recording component 64. In some embodiments the electrical recording component 64 includes an electrode configured to detect electrical signals from the patient. The electrodes can include surface-contact electrodes or inserted electrodes. Surface-contact electrodes can be placed on the skin while inserted electrodes are placed within tissue. Inserted electrodes are generally either wire electrodes or needle electrodes. A possible configuration of an electrode is a monopolar configuration which, in some embodiments, includes one insulated wire in a cannula. The wire tip is exposed and acts as the detection surface. The tip detects electrical potential at the point and the signal is then compared to a reference electrode, which is located usually elsewhere and is chosen because the reference is electrically silent or only detects signals unrelated to the target tissue. An example of a reference electrode is a surface-contact electrode on a region distal to the target tissue. A drawback of a monopolar configuration is the potential for unwanted or confounding signals in the vicinity of the detection tip.

Another possible configuration of the electrode of the electrical recording component 64 is a bipolar configuration. A bipolar configuration contains a first wire with a bare tip that acts as a detection surface. The bipolar configuration also contains a second wire in the cannula that provides a second detection surface. The two surfaces are used to detect two electrical potentials in target tissue and both are compared to a reference electrode. The difference of the two electrodes is eliminated as that difference is likely due to noise or other unwanted signals.

A display component 66 operates to generate a display for the physician or other care provider. In some embodiments the display component 66 displays the detected EMG or MUAP signals. An example embodiment of the display component is a display connected to a series of modules, which enables the detected signal to be viewed by the physician performing the procedure. The modules effectively act as filters to convert the signal from the tissue to the electrodes and ultimately to an amplifier and recorder, which then displays the signal onto a screen. Other examples of modules to be used in the display component include a pre-amplifier, high frequency and low frequency band filters, an analog to digital converter, a common mode rejection amplification system, and a computing device that displays the obtained signals. The computing device may executes program code relating to EMG and general control features such as reading and/or writing transformed data from/to the storage component and/or the I/O component for further processing, providing a communications link between each of the components in the computer system, and a communication module that allows the physician to interact or communicate with the display system. To this extent, the display component 66 can manage a set of interfaces (e.g., graphical user interface(s) and the like) that enable the physician and/or users to interact with the display component 66. Furthermore, the display component can manage data (e.g., store, retrieve, create, transfer) with other computing devices and/or across a communication network.

The temperature sensing component 68 operates to sense rapid changes in thermal conditions including drops or rises in temperature. The temperature sensing component 68 can include a variety of additional components modules such as electronic components (e.g., resistors, diodes, and thermocouples). Temperature sensing component 68 may in some embodiments employ one or more of two types of resistors: a negative temperature coefficient (NTC) resistor or a positive temperature coefficient (PTC) resistor, or other components. A PTC may be comprised of a metal alloy or a combination of metal alloy such as platinum or rhodium-iron. NTC resistors may be comprised of alloys such as germanium, carbon-glass, or ruthenium oxide.

In general, the cryogenic substance delivery component 69 operates to deliver cryogenic substance to the cryoprobe needle 40 for the cryoablation procedure. The cryogenic substance delivery component 69 provide the inflow of high pressure (usually from 650-800 psi) cryogenic substance into the fixed volume chambers in the cryoprobe needle 40. Examples of cryogenic substance include liquid nitrogen, nitrous oxide, or carbon dioxide. The rate of inflow is determined by the diameter of the cryoprobe needle and ranges from about 8-15 liters/minute. As the cryogenic substance travels from the smaller inflow chamber and exits through the larger exhaust chamber, there is a decrease of pressure and a rapid drop in temperature. This will cool the conducting surface of the cryoprobe needle tip and subsequently any tissue in contact with the cryoprobe needle tip. An ice ball or cryolesion is formed on any tissue in contact with the cryoprobe needle tip. An embodiment of the cryogenic substance delivery component 69 may also have flow regulators, connectors, and flow valves.

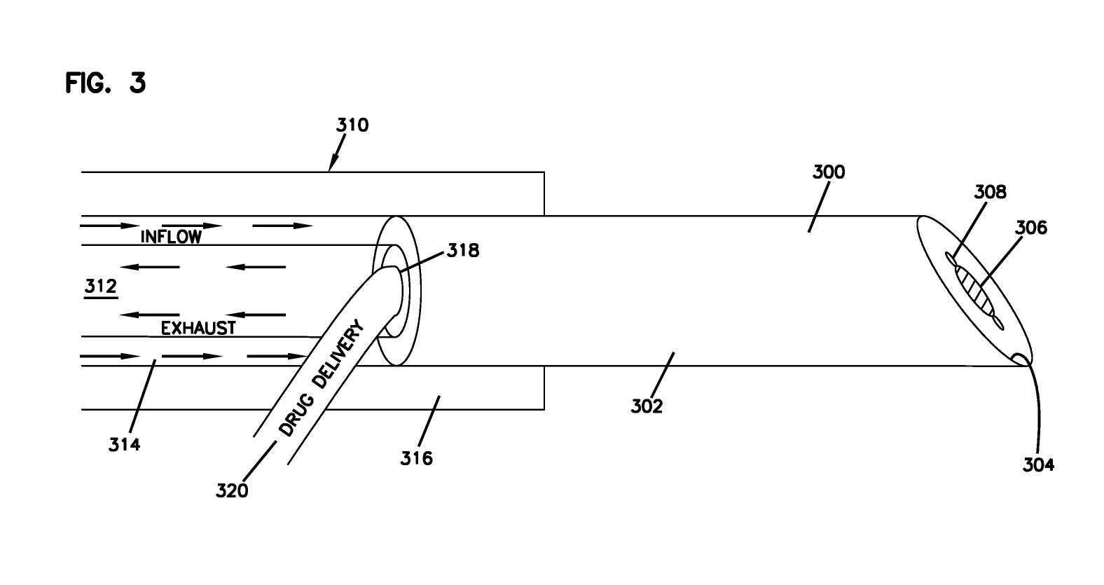

FIG. 3 is a schematic diagram of an example cryoprobe needle 300, which is an example of the cryoprobe needle 40 shown in FIGS. 1 and 2. An example embodiment of the cryoprobe needle 300 comprises a needle shaft 302 and a needle tip 304 at the distal end of the needle shaft 302. In an embodiment, the needle tip 304 has a drug delivery opening 306 that allows local anesthetic to be delivered to the targeted nerve. Also positioned adjacent to the drug delivery opening 306 are one or more stimulating electrodes 308, which may be electrically connected by one or more electrical conductors routed through one or more apertures in the needle 300, to the electrical stimulating component 62.

In some embodiments the proximal end of the needle shaft 302 is coupled to a cryogenic tube 310. In an embodiment, the cryogenic tube 310 includes at least two chambers: an exhaust chamber 312 and an inflow chamber 314. The exhaust chamber 312 allows for the exhaust of cryogenic substance whereas the inflow chamber 314 allows for the inflow of cryogenic substance. A throttling device (such as a valve) may connect the two chambers in some embodiments to control or moderate the flow of the cryogenic substance. In some embodiments the exhaust chamber 312 is substantially larger in volume as compared to the inflow chamber 314. The difference in chamber volume allows for the rapid decrease in pressure as substance flows from the inflow chamber 314 to the exhaust chamber 312. Additionally, the cryogenic tube 310 may be insulated with an insulating layer 316, which maintains the cryogenic tube 310 at a stable and constant temperature. In another embodiment, the cryogenic tube 310 may be adapted to fit over the inner shaft tube 318 of the needle shaft, which creates a path for the delivery of drugs such as local anesthetics. The inner shaft tube 318 connects to a drug delivery system or device or syringe 320. In an embodiment, a leuer lock adapter may be configured to fit over the proximal edge of the inner shaft tube 318.

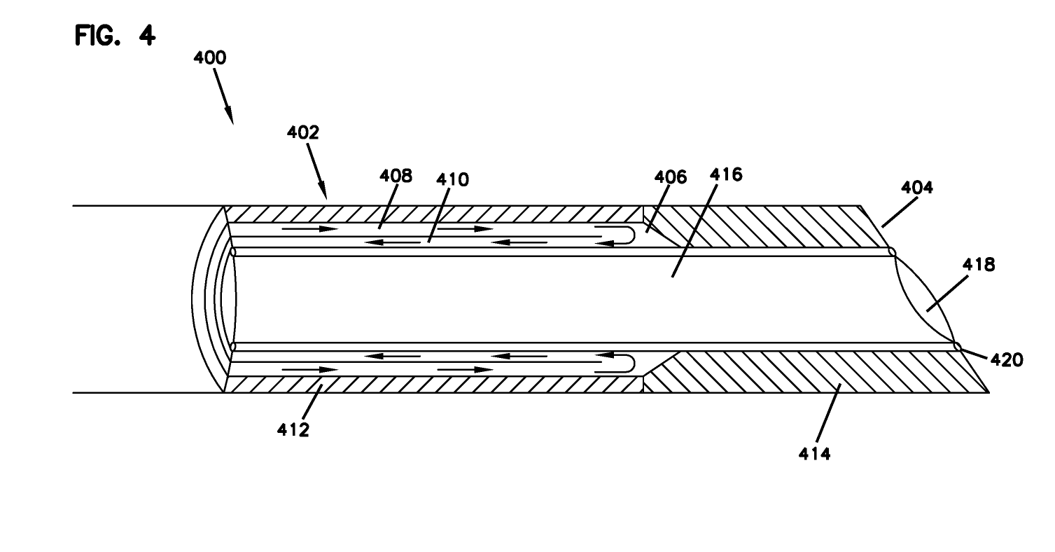

FIG. 4 is a schematic longitudinal cross section of an example cyroprobe needle 400. In some embodiments the needle 400 is an example of the needle 300, and/or an example of the needle 40 discussed herein. In some embodiments, the cryoprobe needle 400 is a closed system that comprises a needle shaft 402 and a cryoprobe tip 404. In some embodiments both the needle shaft 402 and the cryoprobe tip 404 comprise multiple concentric tubes. In an embodiment, the needle shaft 402 also has an outer shaft tube 406 that runs to the proximal end of the cryoprobe tip 404. The outer shaft tube 406 further comprises two chambers; one chamber is reserved for the inflow of cryogenic substance while the other chamber is reserved for the exhaust of cryogenic substance. A throttling device (e.g. valve) may connect the two chambers. For example, cryogenic substance like nitrous oxide will flow from the inflow chamber 408 to the proximal end of the cryoprobe tip 404 and then proceed to exit through the exhaust chamber 410 and back into the cryogenic tube 310 illustrated in FIG. 3. This creates a unidirectional flow of the cryogenic substance that leads to rapid cooling of the cryoprobe tip 404. The needle shaft 402 also has an outer insulating layer 412 that ensures that the needle shaft maintains a moderate temperate as the cryogenic substance travels through the inflow chamber 408 and the exhaust chamber 410. This ensures that the needle shaft remains comfortable to touch and manage.

The cryoprobe tip 404 is positioned relative to the needle shaft 402 and the outer shaft tube 406 in such a way that the inflow and exhaust of cryogenic substance will cause a change in pressure within the chambers and lead to cooling of the chambers. The change in temperature inside the inflow chamber 408 and exhaust chambers 410 will consequently lead to a rapid cooling of the conducting surface 414 of the cryoprobe tip 404. Any tissue in contact with the conducting surface 414 will freeze upon contact.

The needle shaft 402 also comprises an inner shaft tube 416 that runs through the needle shaft 402 and terminates at the drug opening 418. In this manner, the inner shaft tube 416 allows for the delivery and injection of drugs from a drug delivery system at a targeted nerve. The inner shaft tube is insulated from the remainder of the needle shaft 402 and cryoprobe tip 404. An insulating material surrounds the inner shaft tube and thereby prevents any drug or fluid traveling in the inner shaft tube 416 from freezing.

The cryoprobe tip 404 comprises a conducting surface 414 such as sterling steel or another metal alloy. In an embodiment, the cryoprobe tip 404 has sharp edges that allow the user or physician to pierce or penetrate tissue such as skin, fat, connective tissue, muscle, and nerve. In another embodiment, the cryoprobe tip 404 may comprise of blunt or rounded edges that allow repositioning of the cryoprobe tip 404 without risk of unintentional penetration or piercing of adjacent tissue. In another embodiment, the cryoprobe tip 404 may comprise of angled sharp edges, pointed sharp edges, or beveled sharp edges allow repositioning of the cryoprobe tip 404.

In another embodiment, the cryoprobe tip 404 has at least two insulated cannulas 420 for the stimulating electrodes. The insulated cannulas may run through both the needle shaft 402 and the cryoprobe tip 404. In various embodiments, stimulating electrodes may comprise of either a needle or wire electrode. As noted above, a wire electrode is generally fine and easily implanted and withdrawn from tissue, and thereby elicits minimal pain when inserting into the tissue. A needle electrode, in contrast, is generally inserted in the tissue throughout the duration of the exam and elicits a great pain response. Example embodiments of a wire based electrical stimulating component include wire electrodes made from small diameter, non-oxidizing, and stiff wire covered with insulation. The wire may be composed from platinum, silver, nickel, or chromium alloy.

The cannulas 420 for the stimulating electrodes may also be insulated. The insulating material surrounds cannulas 420 and thereby prevents the wire or needle electrodes from freezing during a cryoablation procedure. The insulation may be composed from nylon, polyurethane, or Teflon or a number of other insulating materials, polymers, fabric, or substance.

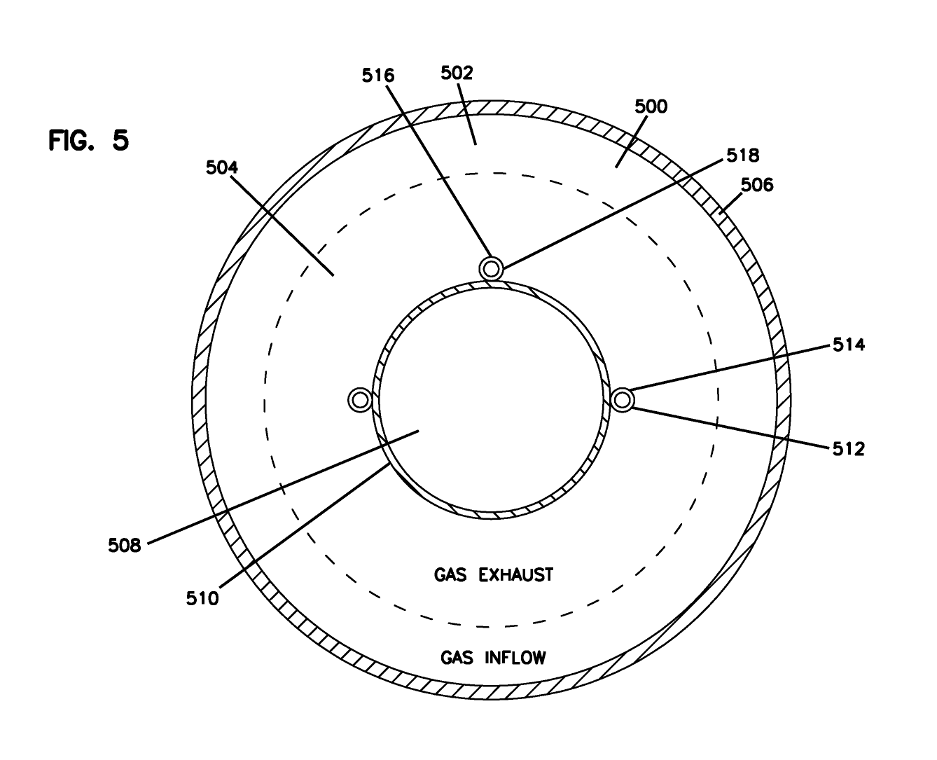

FIG. 5 is a circular cross section of the needle shaft 402 according to embodiments. In an embodiment, the needle shaft 402 comprises multiple concentric tubes. In an embodiment, the needle shaft 402 also has an outer shaft tube 500 that runs to the proximal end of the cryoprobe tip 404. The outer shaft tube 500 further comprises two chambers; one chamber is reserved for the inflow of cryogenic substance while the other chamber is reserved for the exhaust of cryogenic substance. A throttling device (e.g. valve) may connect the two chambers. For example, cryogenic substance like nitrous oxide will flow from the inflow chamber 502 to the proximal end of the cryoprobe tip 404 and then proceed to exit through the exhaust chamber 504 and back into the cryogenic tube 310 illustrated in FIG. 3. This creates a unidirectional flow of the cryogenic substance that leads to rapid cooling of the cryoprobe tip 404. The needle shaft 402 also has an outer insulating layer 506 that ensures that the needle shaft maintains a moderate temperate as the cryogenic substance travels through the inflow chamber 502 and the exhaust chamber 504. This ensures that the needle shaft remains comfortable to touch and manage.

The needle shaft 402 also comprises an inner shaft tube 508 that runs through and to the distal end of the cryoprobe tip 404. The inner shaft tube 508 connects to the drug opening 418. In this manner, the inner shaft tube 508 allows for the delivery of drugs from a drug delivery system through the needle shaft 402 and cryoprobe tip 404 to the targeted nerve. The inner shaft tube is insulated from the remainder of the needle shaft 402 and cryoprobe tip 404. An insulating material 510 surrounds the inner shaft tube 508 and thereby prevents any drug or fluid traveling in the inner shaft tube 508 from freezing.

As stated earlier, the cryoprobe needle has at least two insulated cannulas 512 for the stimulating electrodes. The cannulas 512 for the stimulating electrodes may also be insulated. The insulating material 514 surrounds cannulas 512 and thereby prevents the wire or needle electrodes from freezing during a cryoablation procedure. The insulation may be composed from nylon, polyurethane, or Teflon or a number of other insulating materials, polymers, fabric, or substance.

In another embodiment, the cryoprobe needle has a thermocouple device 518 that runs along both the needle shaft 402 and the cryoprobe tip 404. The thermocouple device 518 allows for measuring the temperature of the target nerve or tissue. The thermocouple device may be linked to the temperature sensing component 68, described in detail above and in FIG. 2. As noted, the temperature sensing component may comprise numerous additional components modules such as resistors, diodes, and thermocouples. In an embodiment, the thermocouple device 518 may also be insulated as it runs along the needle shaft. The insulating material 516 surrounds the thermocouple device 518 and thereby prevents it freezing during a cryoablation procedure. The insulation may be composed from nylon, polyurethane, or Teflon or a number of other insulating materials, polymers, fabric, or substance.

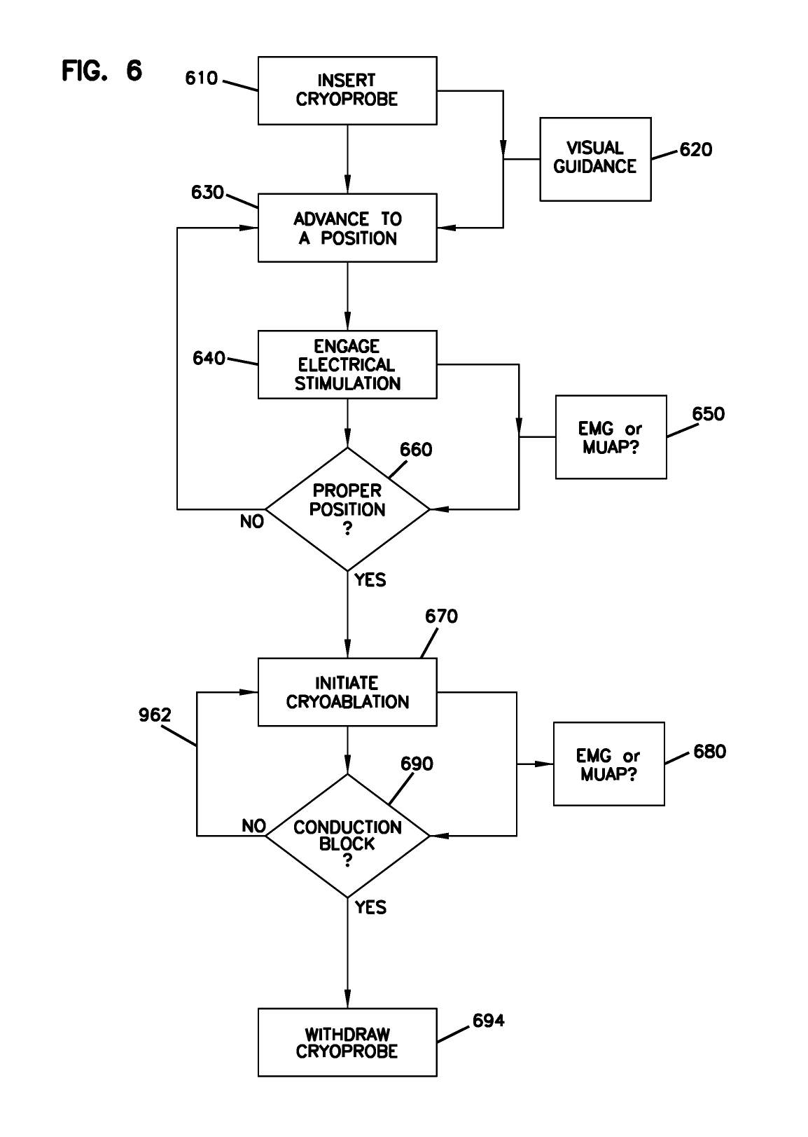

FIG. 6 is a flowchart illustrating an example method of using the cryoablation system. While primarily shown and described herein as a method for blocking the conduction of a target nerve as part of a cryoablation procedure, it is understood that aspects of the disclosure further provide various alternative embodiments. In FIG. 6, a healthcare provider inserts into a recently anesthetized skin the sharp distal needle tip of the cryoprobe needle 610. Using the visual guidance instrument 620, the healthcare provider can approximate the position of the cryoprobe needle to a first position 630. Examples of visual guidance instruments include an ultrasound device or a fluoroscope. Once at the desired position, the healthcare provider can then engage the electrical stimulating component 640 (50-100 Hz for sensory nerves and 1-2 Hz for motor nerves). EMG or MUAP recordings can then be obtained from the targeted muscle using the electrical recording component 650. An example embodiment of an electrical recording component is a bipolar, 90-degree hook electrodes placed on the muscles associated with the targeted nerve. Stimulation is intermittently performed and the signal strength of the electrical recording component is observed. As the cryoprobe needle is positioned closer to the targeted nerve, the strength of the electrical recording signal strength increases. A maximum signal suggests an optimal placement of the cryoprobe needle next to the targeted nerve. Once the optimal location is determined, cryoablation treatment can be initiated.

In FIG. 6, the cryoablation treatment is initiated by starting the inflow of high pressure (usually from 650-800 psi) cryogenic substance into the fixed volume chambers in the cryoprobe needle 670. Examples of cryogenic substance include liquid nitrogen, nitrous oxide, or carbon dioxide. The rate of inflow is determined by the diameter of the cryoprobe needle and ranges from about 8-15 liters/minute. As the cryogenic substance travels from the smaller inflow chamber and exits through the larger exhaust chamber, there is a decrease of pressure and a rapid drop in temperature. This will subsequently cool the conducting surface of the cryoprobe needle tip and subsequently any tissue in contact with the cryoprobe needle tip. An ice ball or cryolesion is formed on any tissue in contact with the cryoprobe needle tip.

The size of the ice ball formed from the tissue depends on a number of factors including the size of the of the cryoprobe needle tip, the duration of exposure, and whether the cryoprobe tip is placed near the vicinity of a blood vessel, which may function as a heat sink. Furthermore, substance flow is controlled by an attached console which has flow regulators, connectors, and may be associated with the temperature sensing component.

As noted above, freezing nerve tissue may either reversibly interrupt the conduction of action potentials if the targeted temperatures are controlled between −20 and −100 degrees Celsius. When temperatures are between −20 and −100 degrees Celsius, the nerve axon is affected (resulting in conduction block of an action potential) but the perineurium and epineurium remain intact, which enables nerve regeneration. At these temperatures, the nerve will initially degenerate at a region distal to the lesion (Wallerian degeneration) but will subsequently regenerate using the endoneurium and perineurim as scaffolds at a rate of 2-5 mm/day.

Cryoablation may also permanently damage the nerve tissue if the target temperature is below −140 degrees Celsius. At temperatures below −140 degrees Celsius, both axons and all surrounding connective tissue, including endoneurium, perineuirum, and epineurium, are frozen and destroyed. This prevents nerve regeneration and therefore the nerve tissue is irreversibly damaged.

Two to three minute freezing cycles 692 are performed with a thawing cycle in between freezing cycles. The duration of a freezing cycle is approximately 2-3 minutes each cycle. As used herein, it is understood that a thawing cycle is a cycle permitting the targeted tissue to thaw after a freezing cycle. The duration of a thawing cycle is about 30 seconds. Thawing between freezing cycles may increase the size of the ice ball or cryolesion, thereby making the cryolesion more effective.

After the freezing and thawing cycles, the healthcare provider may again initiate the electrical stimulating component (50-100 Hz for sensory nerves and 1-2 Hz for motor nerves). EMG or MUAP recordings can then be obtained from the targeted muscle using the electrical recording component 680. The observed EMG or MUAP can confirm or deny whether the targeted nerve was successfully ablated with cryoablation. Absent or diminished EMG or MUAP signal suggests that the targeted nerve was successfully ablated. This also further verifies that the targeted nerve was indeed physiologically and anatomically correctly identified. Prior to withdrawing the cryoprobe needle 694, 1-2 cc of a local anesthetic may again be injected to provide post-procedure analgesia. Examples of local anesthetic include bupivacaine 0.5%.

The various embodiments described above are provided by way of illustration only and should not be construed to limit the claims attached hereto. Those skilled in the art will readily recognize various modifications and changes that may be made without following the example embodiments and applications illustrated and described herein, and without departing from the true spirit and scope of the following claims.

1. A needle for applying cryoablation to a peripheral nerve in a patient, the needle comprising:
an insulated needle shaft, the insulated needle shaft comprising:
an inner shaft tube;
at least two cannula leads;
an outer shaft tube, wherein the outer shaft tube comprises an inflow chamber and an exhaust chamber, the inflow chamber configured to allow inflow of a cryogenic substance and the exhaust chamber configured to allow exhaust of the cryogenic substance; and
a cryoprobe tip connected to the distal end of the insulated needle shaft, the cryoprobe tip comprising:
a conducting surface configured to rapidly cool;
at least two apertures connected to the at least two cannula leads; and
an opening connected to the inner shaft.

2. The needle of claim 1, further comprising:
a thermocouple junction running along the length of the insulated needle shaft and the cryoprobe tip.

3. The needle of claim 1, wherein the cryoprobe tip is a sharp tip configured to perforate a skin of the patient.

4. The needle of claim 1, wherein the cannula leads are in a bipolar configuration.

5. The needle of claim 1, further comprising:
a leur lock adapter positioned at a proximal edge of the insulated needle shaft, wherein the leur lock adapter is configured for attachment of a syringe for drug delivery.

6. The needle of claim 1, wherein the inflow chamber and exhaust chamber are separated by a throttling device.

7. A system for blocking the conduction of a targeted peripheral nerve in a patient, the system comprising:
a cryoprobe needle, wherein the cyroprobe needle comprises:
an inner channel configured to allow injection of a local anesthetic;
an insulated needle shaft with an inflow chamber and an exhaust chamber, the inflow chamber configured to allow inflow of a cryogenic substance and the exhaust chamber configured to allow exhaust of the cryogenic substance;
at least two cannula leads;
a drug delivery device for the delivery of drug; and
a control system for generating electrical stimulation signals and measuring data corresponding to EMG or motor unit action potentials.

8. The system of claim 7, wherein the control system further comprises a display component for displaying data corresponding to EMG or motor unit action potentials.

9. The system of claim 7, wherein the control system further comprises a temperature sensing component to measure and record temperature changes in the targeted peripheral nerves.

10. The system of claim 7, wherein the control system further comprises cryogenic substance delivery component to deliver cryogenic substance to the cryoprobe needle.

11. A method for blocking the conduction of targeted peripheral nerve in a patient, the method comprising:
inserting a cryoprobe through a skin of a patient and into a first position toward a targeted peripheral nerve;
generating with an electrical stimulator a first signal at a conducting tip of the cryoprobe, wherein the first signal stimulates the targeted peripheral nerve to innervate a targeted muscle;
receiving a second signal from the targeted muscle at a recording tip of the cryoprobe, the second signal being associated with muscle activity of the targeted muscle; and
generating with the cryoprobe a cryolesion on the targeted peripheral nerve;
generating with an electrical stimulator a third signal at a conducting tip of the cryoprobe, wherein the third signal stimulates the targeted peripheral nerve at the location of the cryolesion; and
receiving a fourth signal from the muscle at a recording tip of the second needle, the fourth signal confirming successful ablation of the peripheral nerve.

12. A needle for applying cryoablation to a peripheral nerve in a patient, the needle comprising:
an insulated needle shaft, the insulated needle shaft comprising:
an inner shaft tube;
an outer shaft tube, wherein the outer shaft tube comprises an inflow chamber and an exhaust chamber, the inflow chamber configured to allow inflow of a cryogenic substance and the exhaust chamber configured to allow exhaust of the cryogenic substance; and
a cryoprobe tip with a conducting surface and an opening connected to inject drugs.
